(19) United States
(12) Patent Application Publication
(10) Pub. No.: US 2005/0268365 A1
(43) Pub. Date: Dec. 8, 2005

(54) Baseball catcher's protective sleeve

(76) Inventor: Steven Sandebeck, Chestertown, MD (US)

Correspondence Address: Royal W. Craig;Law Offices of Royal W. Craig, Suite 153, 10 N. Calvert Street, Baltimore, MD, 21202, US

(21) Appl. No.: 11/099,197

(22) Filed: Apr. 5, 2005

Related U.S. Application Data
(60) Provisional application No. 60/560,010, filed on Apr. 5, 2004.

(51) Int. Cl. A61F 9/00

(52) U.S. Cl. 2/16

(57) ABSTRACT

A baseball catcher's protective sleeve to shield the inner forearm and wrist from short hop and wild pitches. The baseball catcher's protective sleeve is particularly suited for children, and also particularly suited for training and practice. The sleeve comprises an inner and outer tubular shell formed of a stretchable, moisture-absorbent fabric and sewn together with a soft pad conforming to the inner forearm sewn there between in a segmented manner for flexibility and protection. The protective sleeve fits securely around the forearm without slipping or causing discomfort. A tongue extends from the distal end of the sleeve and extends over the distal end of the wearer's palm to extend protection to both the gloved and throwing hand. The baseball catcher's protective sleeve is comfortable, lightweight, flexible, moisture-absorbent, and sufficiently padded to shield the inner forearm and wrist from short hop or wild pitches.

### CROSS REFERENCE TO RELATED APPLICATIONS

The present application derives priority from U.S. Provisional Patent Application 60/560,010 for “BASEBALL CATCHER's PROTECTIVE SLEEVE”, filed Apr. 5, 2004.

### BACKGROUND OF THE INVENTION

1. Field of the Invention.

The present invention relates to baseball protective equipment and, more particularly, to a baseball catcher's protective sleeve that shields the inner forearm and wrist against short hops and wild pitches, during play, and especially during training and instructional practice exercises.

2. Description of the Background.

Protective safety equipment is recommended and used in many different sports, including baseball, football, ice hockey, lacrosse, etc. The purpose of protective equipment is to prevent and/or reduce the severity of sports-related injuries. There are many types of protective equipment, generally designed for specific sports and often to protect from specific injury. The use of protective equipment in any given sport may be advocated and/or promoted by the government, safety groups, health professionals, sports associations, or other special interest groups. There are several national organizations that have developed standards for safety equipment, such as the National Operating Committee on Standards for Athletic Equipment (NOCSAE) at www.nocsae.org.

In June 1996, the U.S. Consumer Product Safety Commission (CPSC) announced that baseball safety equipment could significantly reduce the amount and severity of 58,000 (or almost thirty-six percent of) baseball-related injuries to children every year. Baseball, softball, and teeball are among the most popular sports in the United States, with an estimated 6 million children ages 5 to 14 participating in organized leagues and 13 million children playing unorganized games. In 1995, 162,100 children were treated in hospital emergency rooms for baseball-related injuries. (See CPSC Releases Study of Protective Equipment for Baseball—Release #96-140, at www.cpsc.gov/cpscpub/prerel/prhtml96/96140.html.) Of the 162,100 hospital emergency room treated injuries in 1995, almost seventy-five percent of the injuries occurred to older children ages 10-14, which represents about half of the total number of children playing baseball. CPSC considered about thirty-three percent of the total injuries as severe, including fractures, concussions, internal injuries, and dental injuries. The remaining 67 percent less severe injuries include contusions, abrasions, lacerations, strains, and sprains. More than 50 percent of the injured children under 11 years old that were injured while playing baseball sustained injuries to the head and neck area, while a larger percentage of older children sustained injuries to their arms and legs.

Catchers are particularly susceptible to injury due to their proximity to the batter and the batter's box. Because of this, catchers generally wear a specific catcher's headgear, face mask, catcher's mitt, chest protector, and padded leg guards However, the inner forearm remains exposed, despite being particularly susceptible to wild pitches and short hops. This is especially true of child catchers, who tend to be intimidated by wild pitches and instinctively move out of the way or shield themselves with their forearms, rather than catching the wild pitch. The inner forearms should have some protection.

Because of the need to protect athletes, particularly children, from sports-related injuries, many types of protective equipment are well-demonstrated in the prior art.

For example, U.S. Pat. No. 5,669,080 to Culton discloses a protective apparatus against baseball pitching injury, which is a one-piece shock-absorbing vest contoured to protect the chest, shoulder and upper arm areas of the wearer. The protective vest is made from a rubber-like pad that absorbs shock and resists moisture, covered on the front with a breathable fabric and on the back with a moisture-wicking fabric.

U.S. Pat. No. 4,989,265 to Nipper et al. shows a chest protector shoulder pad formed of a layer of closed cell elastomeric foam having a regularly bi-directional convoluted surface.

U.S. Pat. No. 5,887,277 to Lohman discloses a protective elbow pad formed from a layer of flexible, resilient padding material. The forward section overlays the forearm and the rearward section overlays the upper arm, with a hole between to receive the wearer's elbow.

U.S. Pat. No. 6,553,579 to Gillen et al. shows a removable protective shoulder pad that protects the shoulder and upper arm. The protective pad, which can secured to a protective body garment, comprises an outer layer or layers of rigid, semi-rigid or semi-flexible material and an inner layer or layers of flexible, semi-flexible or semi-rigid material.

U.S. Pat. No. 5,065,158 to Lutz shows a baseball catcher's hand protector for the throwing hand and U.S. Pat. No. 4,987,611 to Maye shows a protective pad to be worn under a catcher's mitt or first baseman's glove.

Other examples of protective equipment for the arm, but not specifically made for sports, include U.S. Pat. No. 6,079,055 to Mencel, which shows a protective shoulder pad for supporting a load, having a band forming an underarm loop and a pair of body straps, U.S. Pat. No. 6,240,565 to Spear, which shows a waiter's hot-plate arm-shield gauntlet safety pad specifically configured to protect a professional waiter's arm from heat injuries, and U.S. Pat. No. 4,884,297 to Triche, which shows an arm protector for the arm of a welder, which includes both padding and insulation to protect from heat.

Other athletic protective devices have evolved that are specifically for arm protection during sports activities. For example, U.S. Pat. No. 4,344,189 to Futere, et al. discloses one embodiment of a limb protector for athletes that is an arm guard which extends from the wearer's elbow to his wrist. The device is configured to protect the outer forearm. The arm guard includes a tapered sleeve-like element which is sufficiently elastic to be worn tightly about the arm. A pocket in the sleeve-like element holds a protective pad. U.S. Pat. No. 6,374,408 to Tomlinson et al. shows a protective athletic pad apparatus that extends across the elbow and along the front and sides of the forearm to protect the wearer from abrasion, cuts and bruises while actively engaged in a sports activity. The pad is adjustably secured with hook and loop fasteners.

Although all of the aforementioned examples address protective equipment, either particularly for sports use or arm related, or both, they are either not flexible and too cumbersome, do not provide sufficient protection to prevent injury to a catcher's forearm, and/or are apt to move and shift during active play. Moreover, none of the prior art devices are particularly suited to be worn by a baseball catcher to protect his inner forearm and wrist while blocking balls.

Therefore, it would be advantageous over the prior art to provide a baseball catcher's protective cylindrical-shaped sleeve that is comfortable, light-weight, stretchable, flexible, moisture-absorbent, and sufficiently padded half way around the circumference to shield the inner forearm from short hops or wild pitches.

### SUMMARY OF THE INVENTION

It is, therefore, the primary object of the present invention to provide a baseball catcher's protective sleeve to shield the inner forearm from short hop or wild pitches.

It is another object of the present invention to provide a baseball catcher's protective sleeve to shield the wrist and heel of the hand.

It is another object of the present invention to provide a baseball catcher's protective sleeve that is both comfortable and flexible.

It is still another object to provide a baseball catcher's protective sleeve that is lightweight and stretchable to conform to the wearer's forearm.

It is another object to provide a baseball catcher's protective sleeve that is sufficiently padded to protect from injury.

It is yet another object to provide a baseball catcher's protective sleeve that absorbs moisture.

It is another object to provide a baseball catcher's protective sleeve that is particularly suited for children.

The above objects are accomplished by providing a baseball catcher's protective sleeve to be worn on the forearm, comprising a cylindrical-shaped sleeve formed from an outer and inner shell of stretchable, light, moisture-absorbent flexible fabric enclosing soft segmented pads on the forearm side of the sleeve, for force absorption and flexibility. The sleeve fits securely around the forearm without slipping or causing discomfort. A tongue extends from the distal end of the padded portion of the sleeve and extends over the wearer's wrist and slightly over the palm to protect the wrist and heel of the hand.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which:

FIG. 1 is a top perspective view illustrating the baseball catcher's protective sleeve of the present invention.

FIG. 2 is a cross-sectional view of the baseball catcher's protective sleeve of FIG. 1.

FIG. 3 is a side perspective illustration of the baseball catcher's sleeve of the present invention.

FIG. 4 is a perspective illustration of the baseball catcher's sleeve of the present invention in use.

### DETAILED DESCRIPTION OF THE PREFERRED EMBODIMENTS

The present invention provides protection to a baseball catcher's inner forearm and wrist from short hop and wild pitches while the catcher is in the blocking position. The invention is particularly suited for children and teens that have a tendency to shy away from wild pitches, and is particularly useful for all ages during practice and training when upwards of 100 balls may be thrown to a catcher.

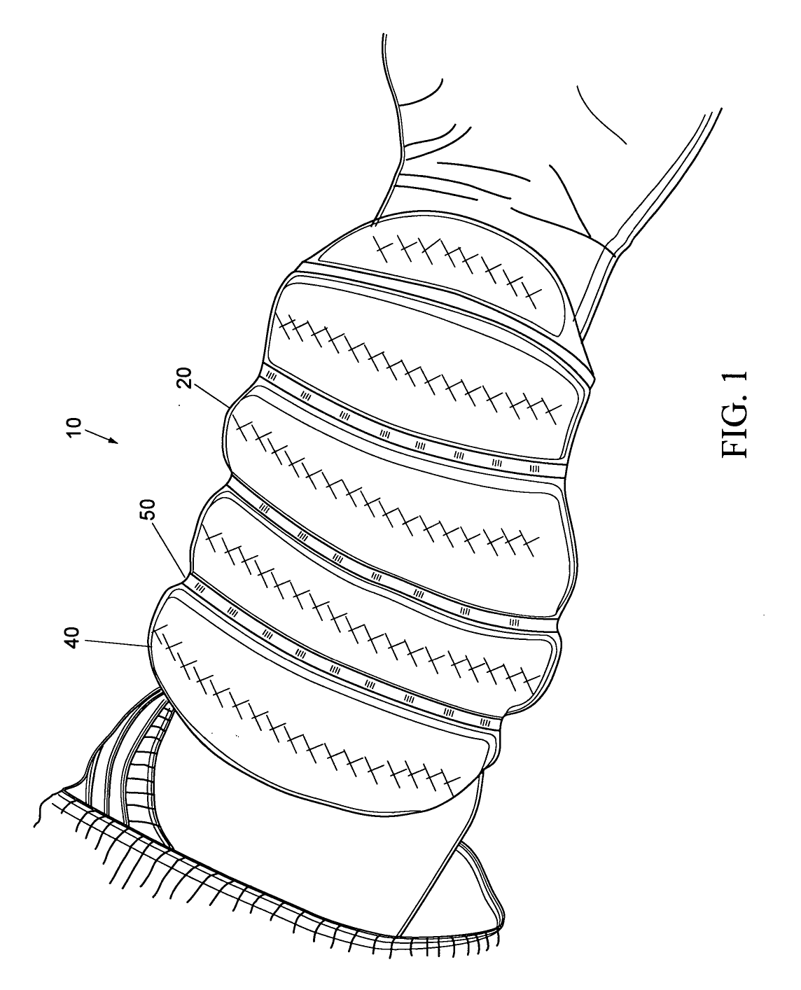

Referring to FIG. 1, the above objects are accomplished by providing a baseball catcher's protective sleeve 10 comprising a stretchable outer shell 20 and inner shell 30 (not shown), with flexible protective padding 25 sewn in between the outer and inner shell 20 and 30, respectively, creating a padded portion 40. The sleeve 10 is cylindrical in shape and fashioned to slip over the catcher's hand and extend over the forearm from the wrist to the elbow, secured comfortably in place with the padded portion of the sleeve 40 covering the inner forearm of the catcher. The portion of the sleeve worn over the outer forearm includes a half-moon cut-out 13 for the elbow to prevent restriction of movement, particularly when the catcher throws the ball. The outer shell 20 and inner shell 30 are made of a lightweight, stretchable, flexible, moisture wicking, absorbent material, preferably a micro-fiber fabric, such as Under Armor®. The micro-fiber stretchable/flexibility allows the sleeve 10 to conform to the catcher's forearm without slipping, shifting or bulkiness, and is not cumbersome or uncomfortable. No awkward straps, flaps, or fasteners are necessary. The catcher merely pulls the sleeve 10 over his hand to extend over the forearm and the sleeve remains in place until the catcher wishes to remove it. Padding 25 is layered between the inner and outer shell 20, 30 and extends the length of the sleeve 40, but only extends half the way around the circumference of the sleeve 40. In use, the sleeve is worn so that the padded portion 40 shields the inner forearm. The padding is conventional blown-foam padding formed from a sheet of particular thickness (between 1/3- 1/2″ inch thickness is preferred) and cut in a trapezoidal pattern of approximately 2″ top width, 3″ bottom width, and 5″ side lengths. The trapezoidal pattern conforms to and covers the inner forearm and the foregoing thickness is calculated to be minimal as necessary to absorb the force of a dirt-pitched ball and shield the arm from injury, and yet thin enough so as not to add weight or flex-resistance. The flexibility is further enhanced by multiple seams that are stitched horizontally across the width of the padded portion 40 of the sleeve 10. The seams serve to hold the pad in place, and also act as hinges to provide flexibility so that the arm can easily move and bend. In the illustrated embodiment five seams are sewn approximately two to three inches apart, albeit the distance between and number of seams may be varied to increase or reduce flexibility as a function of the wearer size. Since the sleeve is intended to be used by adults and children, the sizes of the sleeve vary accordingly. Thus, a small child's sleeve may have two or three seams one and one half inches apart, while a large adult's sleeve may have five seams two and one half inches apart. In any case, at least two seams of stitching should be sewn radially about said cylindrical sleeve through the trapezoidal pad.

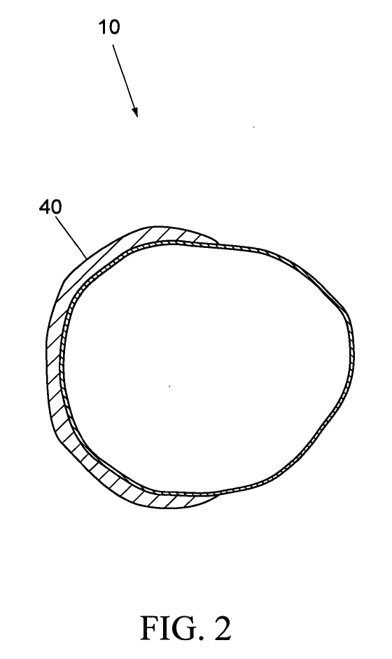

FIG. 2 is a cross-sectional view of the baseball catcher's protective sleeve 10 of FIG. 1. The pad 25 is sewn in between the inner 30 and outer 20 shell to create a padded portion 40 on the forearm side of the sleeve, which is partitioned by seams 50 sewn widthwise across the sleeve to secure the pad in place, while providing flexibility.

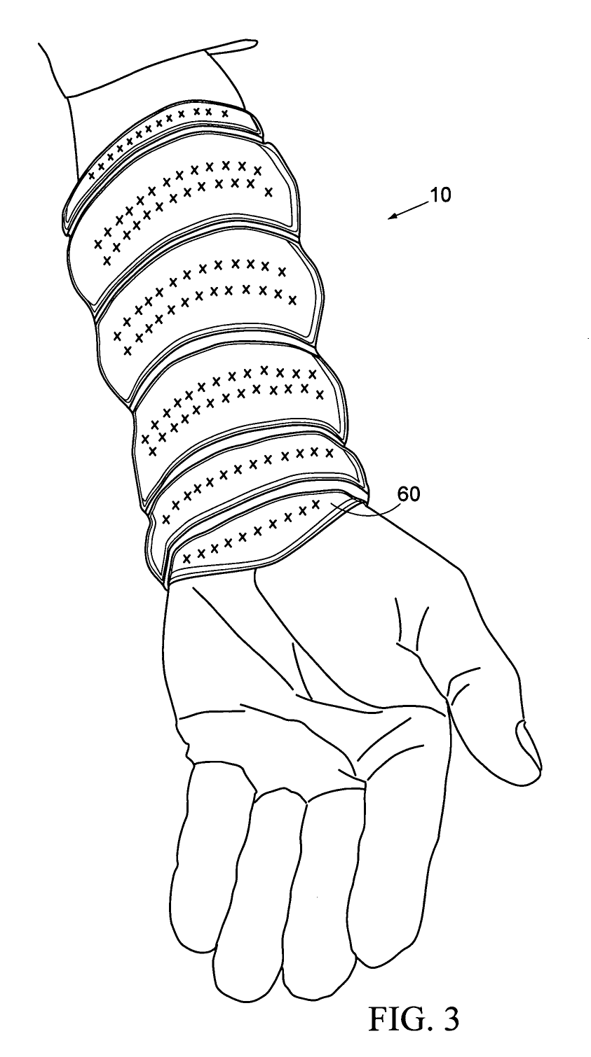

FIG. 3 is a side perspective illustration of the baseball catcher's sleeve of the present invention in place over a catcher's forearm. The outer shell 20 of the padded portion 40 of the sleeve 10 comprises a tongue portion 60 at the distal end of the sleeve that extends down and out to protect the wrist and heel of the catcher's hand. This additional protection shields both the catcher's gloved and throwing hand from the wild pitch that is not caught squarely in the catcher's mitt. On the gloved hand in a wait position, the tongue 60 extends underneath the catcher's mitt. Generally, in the wait position, the catcher's mitt covers the heel of the hand. However, when the hand arches back to catch a ball, the wrist is exposed and the tongue 60 of the sleeve 10 protects the wrist.

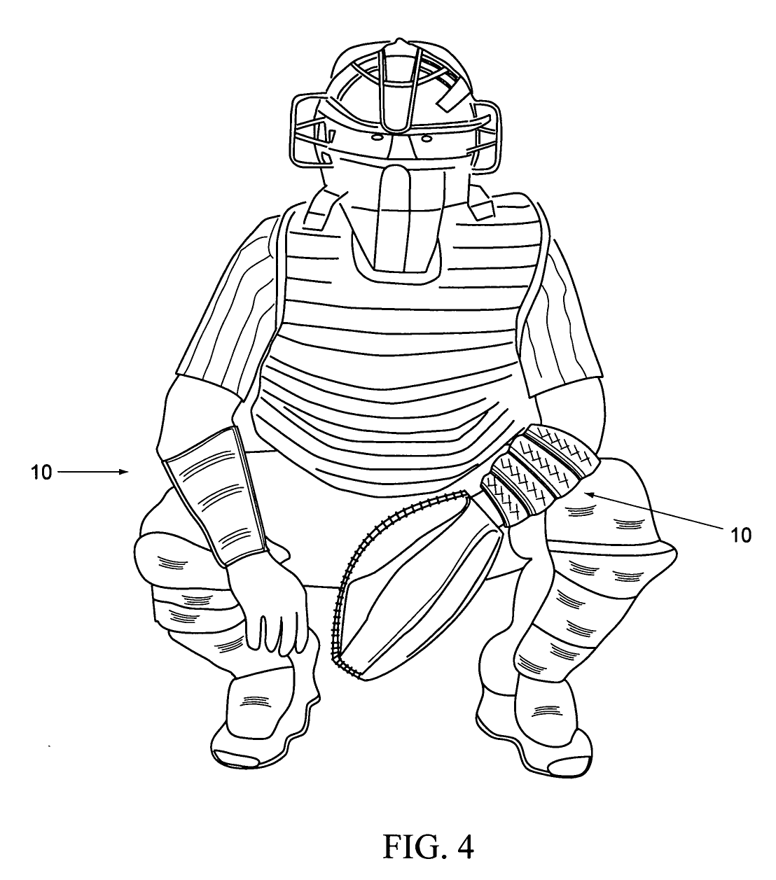

FIG. 4 is a perspective illustration of the baseball catcher's sleeve 10 of the present invention in use. The catcher slips a protective sleeve 10 over each arm and in the normal catcher's stance, the inner forearms and wrists are no longer exposed, but shielded from any short hop or wild pitch. In the FIG. 4 illustration, the catcher's right arm is turned so his palm and inner forearm is facing him, so the padding of the protective sleeve is not shown. The catcher's left hand with the catcher's mitt is facing outward such that the padding portion 40 of the protective sleeve is covering his inner forearm.

In light of the foregoing, it is apparent that the baseball catcher's protective sleeve 10 is particularly designed for use by baseball catchers to eliminate or reduce injuries to the catcher's forearms and wrists from short hop or wild pitches. The sleeve is especially suited for children who may have a tendency to shy away from wild pitches because of the fear of being injured. The sleeve is also particularly suited for instructional and training use when a catcher may have to continuously block short hops and wild pitches.

Having now fully set forth the preferred embodiment and certain optional modifications of the concept underlying the present invention, various other embodiments as well as certain variations and modifications of the embodiment herein shown and described will obviously occur to those skilled in the art upon becoming familiar with said underlying concept. It is to be understood, therefore, that the invention may be practiced otherwise than as specifically set forth in the appended claims.

1. A protective device for a baseball catcher's forearm, comprising:
a sleeve formed from inner and outer shells of lightweight, flexible, moisture wicking microfiber material patterned and sewn to form a cylinder;
a soft absorbent trapezoidal pad sewn into one side of the cylindrical sleeve between said inner and outer shells by stitching through said shells about the periphery of said trapezoidal pad;
a plurality of seams of stitching sewn radially about said cylindrical sleeve and spanning said trapezoidal pad;
wherein said protective device is worn by pulling said sleeve over a forearm with the trapezoidal pad covering an inner forearm to protect the catcher's forearm from short hop and wild pitches while playing or practicing baseball.

2. The baseball catcher's protective sleeve according to claim 1, further comprising a padded tongue extending over a catcher's wrist and partially over the palm.

3. The baseball catcher's protective sleeve according to claim 1, wherein said sleeve is defined by a half-moon-shaped cut-out portion for seating a catcher's elbow to prevent restriction of the elbow.

4. The baseball catcher's protective sleeve according to claim 1, wherein said trapezoidal pad comprises blown foam padding.

5. The baseball catcher's protective sleeve according to claim 1, wherein a thickness of said pad is within a range of from 1/3 to 1/2 inch.

6. A protective device for a baseball catcher's forearm, comprising:
a sleeve formed from inner and outer shells of lightweight, flexible, moisture wicking microfiber material patterned and sewn to form a cylinder;
a soft absorbent pad sewn into one side of the cylindrical sleeve between said inner and outer shells by stitching through said shells about the periphery of said trapezoidal pad, said pad comprising a section of blown-foam of between 1/3- 1/2″ inch thickness and cut in a trapezoidal shape of approximately 2″×3″×5″×5″ to substantially conform to and cover a inner forearm;
at least two parallel seams of stitching sewn radially about said cylindrical sleeve and spanning said trapezoidal pad; and
a padded tongue portion at the distal end of the sleeve extending outward and underneath the catcher's mitt to protect the catcher's wrist;
wherein said protective device is worn by pulling said sleeve over a forearm with the trapezoidal pad covering an inner forearm to protect the catcher's forearm from short hop and wild pitches while playing or practicing baseball.

7. The baseball catcher's protective sleeve according to claim 6, wherein said sleeve is defined by a half-moon-shaped cut-out portion for seating a catcher's elbow to prevent restriction of the elbow.

8. The baseball catcher's protective sleeve according to claim 7, wherein said trapezoidal pad comprises blown foam padding.

9. The baseball catcher's protective sleeve according to claim 8, wherein a thickness of said pad is within a range of from 1/3 to 1/2 inch.
